(19) United States
(12) Patent Application Publication
(10) Pub. No.: US 2025/0383566 A1
(43) Pub. Date: Dec. 18, 2025

(54) COMPOUND, POLYMER, ELECTROCHROMIC DEVICE, AND ELECTRONIC DEVICE

(71) Applicants: Samsung Electronics Co., Ltd., Suwon-si (KR); UIF (University Industry Foundation), Yonsei University, Seoul (KR)

(72) Inventors: Won-Jae JOO, Suwon-si (KR); Eunkyoung KIM, Seoul (KR); Youngnam KWON, Suwon-si (KR); Hwandong JANG, Seoul (KR); Cheolhyun CHO, Seoul (KR)

(73) Assignees: Samsung Electronics Co., Ltd., Suwon-si (KR); UIF (University Industry Foundation), Yonsei University, Seoul (KR)

(21) Appl. No.: 19/236,281

(22) Filed: Jun. 12, 2025

(30) Foreign Application Priority Data
Jun. 13, 2024 (KR) 10-2024-0077148

(51) Int. Cl. G02F 1/1516; C07D 495/04; C07D 519/00; C08G 81/00; C09D 5/24; C09D 5/29; C09D 187/00

(52) U.S. Cl. CPC G02F 1/15165; C07D 495/04; C07D 519/00; C08G 81/00; C09D 5/24; C09D 5/29; C09D 187/005

(57) ABSTRACT

Disclosed are a compound represented by Chemical Formula 1, a polymer of the compound, an electrochromic device, and an electronic device.

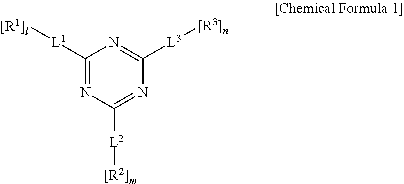

### CROSS-REFERENCE TO RELATED APPLICATION

This application claims priority to and the benefit of Korean Patent Application No. 10-2024-0077148, filed in the Korean Intellectual Property Office on Jun. 13, 2024, the entire contents of which are incorporated herein by reference.

### BACKGROUND

### 1. Field

The present disclosure relates to a compound, a polymer, an electrochromic device, and an electronic device.

### 2. Description of the Related Art

Electrochromic devices may control inflow of light by controlling the color of electronically active materials through electrochemical redox reactions, and thus may be applied to various fields such as optical filters, electronic signage, smart windows, or wearable imaging devices.

### SUMMARY

Some example embodiments provide a compound and/or a polymer thereof capable of implementing effective color control.

Some example embodiments provide an electrochromic device including the polymer.

Some example embodiments provide an electronic device including the compound, the polymer, and/or the electrochromic device.

According to some example embodiments, a compound represented by Chemical Formula 1 is provided.

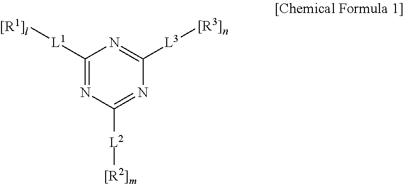

In Chemical Formula 1,
L¹ to L³ each independently may be a single bond, O, S, NR^{a}, a substituted or unsubstituted C1 to C10 oxyalkylene group, a substituted or unsubstituted C1 to C10 thioalkylene group, a substituted or unsubstituted C6 to C30 arylene group, a substituted or unsubstituted divalent C3 to C30 heterocyclic group, or a combination thereof,
R^{a} may be hydrogen, a substituted or unsubstituted C1 to C10 alkyl group, a substituted or unsubstituted C2 to C30 alkenyl group, a substituted or unsubstituted C2 to C30 alkynyl group, a substituted or unsubstituted C1 to C30 alkoxy group, a substituted or unsubstituted C6 to C30 aryl group, a substituted or unsubstituted C3 to C30 heteroaryl group, or a combination thereof,
I, m, and n each independently may be an integer greater than or equal to 1,
R¹ to R³ each independently may be a substituted or unsubstituted C6 to C30 aryl group, a substituted or unsubstituted C3 to C30 heterocyclic group, a substituted or unsubstituted amino group, or a combination thereof, provided that at least one of R¹ to R³ independently may be a group represented by Chemical Formula A,

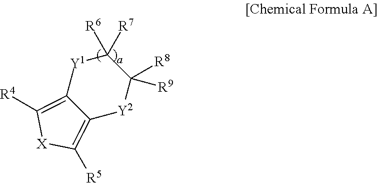

wherein, in Chemical Formula A,
X, Y¹, and Y² each independently may be O, S, Se, or Te,
R⁴ to R⁹ each independently may be hydrogen, a substituted or unsubstituted C1 to C30 alkyl group, a substituted or unsubstituted C2 to C30 alkenyl group, a substituted or unsubstituted C2 to C30 alkynyl group, a substituted or unsubstituted C1 to C30 oxyalkyl group, a substituted or unsubstituted C1 to C30 haloalkyl group, a substituted or unsubstituted C1 to C30 alkoxy group, a halogen, a cyano group, or a linking point with Chemical Formula 1, provided that at least one of R⁴ to R⁹ in Chemical Formula A may be a linking point with Chemical Formula 1,
a may be an integer from 1 to 3, and
if a is 2 or 3, R⁶ may be the same as or different from each other and R⁷ may be the same as or different from each other.

In some embodiments, the compound may have an asymmetric structure in which one or two of L¹ to L³ may be different from a remainder of L¹ to L³ or one or two of R¹ to R³ may be different from a remainder of R¹ to R³.

In some embodiments, the one or two of L¹ to L³ may each independently be O, S, a substituted or unsubstituted C1 to C10 oxyalkylene group or a substituted or unsubstituted C1 to C10 thioalkylene group, and the remainder of L¹ to L³ may each independently be NR^{a}, wherein R^{a} may be hydrogen, a substituted or unsubstituted C1 to C10 alkyl group, a substituted or unsubstituted C2 to C30 alkenyl group, a substituted or unsubstituted C2 to C30 alkynyl group, a substituted or unsubstituted C1 to C30 alkoxy group, a substituted or unsubstituted C6 to C30 aryl group, a substituted or unsubstituted C3 to C30 heteroaryl group, or a combination thereof.

In some embodiments, one or two of L¹ to L³ may each independently be O, S, or NR^{a}, wherein R^{a} may be hydrogen, a substituted or unsubstituted C1 to C10 alkyl group, a substituted or unsubstituted C2 to C30 alkenyl group, a substituted or unsubstituted C2 to C30 alkynyl group, a substituted or unsubstituted C1 to C30 alkoxy group, a substituted or unsubstituted C6 to C30 aryl group, a substituted or unsubstituted C3 to C30 heteroaryl group, or a combination thereof, and the remainder of L¹ to L³ may each independently be a substituted or unsubstituted C1 to C10 oxyalkylene group or a substituted or unsubstituted C1 to C10 thioalkylene group.

In some embodiments, R¹ to R³ may each be different from each other.

In some embodiments, the group represented by Chemical Formula A may be represented by Chemical Formula A-1 or A-2.

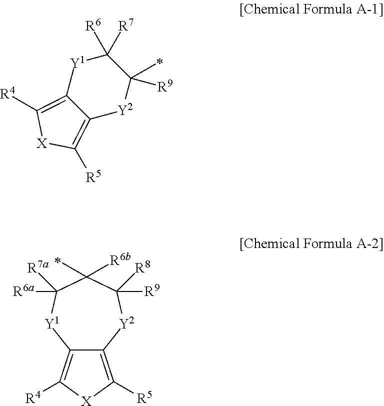

In Chemical Formula A-1 or A-2,
X, Y¹, and Y² each independently may be O, S, Se, or Te,
R⁴, R⁵, R⁶, R^{6a}, R^{6b}, R⁷, R^{7a}, R⁸, and R⁹ each independently may be hydrogen, a substituted or unsubstituted C1 to C30 alkyl group, a substituted or unsubstituted C2 to C30 alkenyl group, a substituted or unsubstituted C2 to C30 alkynyl group, a substituted or unsubstituted C1 to C30 oxyalkyl group, a substituted or unsubstituted C1 to C30 haloalkyl group, a substituted or unsubstituted C1 to C30 alkoxy group, a halogen, a cyano group, or a combination thereof, and
* may be a linking point with Chemical Formula 1.

In some embodiments, in Chemical Formula 1, some of R¹ to R³ may be a group represented by Chemical Formula A-1, and some of R¹ to R³ may be a group represented by Chemical Formula A-2.

In some embodiments, R¹ may be a group represented by Chemical Formula A-1, R² may be a group represented by Chemical Formula A-2, R³ may be a substituted or unsubstituted phenyl group, a substituted or unsubstituted biphenyl group, a substituted or unsubstituted naphthyl group, a substituted or unsubstituted anthracenyl group, a substituted or unsubstituted fluorenyl group, a substituted or unsubstituted triphenylene group, a substituted or unsubstituted carbazolyl group, a substituted or unsubstituted aniline group, a substituted or unsubstituted pyrrole group, a substituted or unsubstituted indole group, a substituted or unsubstituted thiazole group, a substituted or unsubstituted benzothiazole group, a substituted or unsubstituted thiadiazole group, a substituted or unsubstituted benzothiadiazole group, a substituted or unsubstituted imidazole group, a substituted or unsubstituted benzoimidazole group, a substituted or unsubstituted pyrazolyl group, a substituted or unsubstituted phenylenevinylene group, a substituted or unsubstituted triphenylamine group, or a combination thereof.

In some embodiments, in Chemical Formula 1, one or two of R¹ to R³ may be a substituted or unsubstituted phenyl group, a substituted or unsubstituted biphenyl group, a substituted or unsubstituted naphthyl group, a substituted or unsubstituted anthracenyl group, a substituted or unsubstituted fluorenyl group, a substituted or unsubstituted triphenylene group, a substituted or unsubstituted carbazolyl group, a substituted or unsubstituted aniline group, a substituted or unsubstituted pyrrole group, a substituted or unsubstituted indole group, a substituted or unsubstituted thiazole group, a substituted or unsubstituted benzothiazole group, a substituted or unsubstituted thiadiazole group, a substituted or unsubstituted benzothiadiazole group, a substituted or unsubstituted imidazole group, a substituted or unsubstituted benzoimidazole group, a substituted or unsubstituted pyrazolyl group, a substituted or unsubstituted phenylenevinylene group, a substituted or unsubstituted triphenylamine group, or a combination thereof.

According to some example embodiments, a polymer may include a structural unit derived from the compound.

In some embodiments, the polymer may further include a moiety derived from polyethylene glycol, polypropylene glycol, or a combination thereof.

In some embodiments, the polymer may include at least one of R¹, R², or R³ of Chemical Formula 1 as a repeating unit.

According to some example embodiments, a polymer film including the polymer may be provided.

In some embodiments, the polymer film may be configured to reversibly display black or transparency depending on an applied voltage, and a difference in light transmittance depending on the applied voltage may be greater than or equal to about 60% for light of a wavelength of about 500 nm.

According to some example embodiments, an electrochromic device may include a first electrode and a second electrode, an electrochromic layer between the first electrode and the second electrode and including the polymer, and an electrolyte in contact with the electrochromic layer.

The polymer may include a first polymer and a second polymer including different repeating units from each other.

The electrochromic layer may include a first electrochromic layer including the first polymer, and a second electrochromic layer including the second polymer.

The electrochromic device may be configured to reversibly display black or transparency depending on the applied voltage, and a difference in light transmittance depending on the applied voltage may be greater than or equal to about 60% for light of a wavelength of about 500 nm.

According to some example embodiments, an electronic device including the electrochromic device may be provided.

According to example embodiments, a compound capable of effectively controlling electrochemical and optical characteristics may be provided to achieve higher color contrast.

### BRIEF DESCRIPTION OF THE DRAWINGS

FIG. 1 is a cross-sectional view illustrating an example of an electrochromic device according to some example embodiments,

FIGS. 2A and 2B are photographs showing the color change according to the voltage application of the electrochromic device according to Example 1,

FIGS. 3A and 3B are photographs showing the color change according to the voltage application of the electrochromic device according to Comparative Example 1, and

FIG. 4 is a schematic view of an electronic device according to some example embodiments.

### DETAILED DESCRIPTION

Expressions such as “at least one of,” when preceding a list of elements, modify the entire list of elements and do not modify the individual elements of the list. For example, “at least one of A, B, and C,” and similar language (e.g., “at least one selected from the group consisting of A, B, and C”) may be construed as A only, B only, C only, or any combination of two or more of A, B, and C, such as, for instance, ABC, AB, BC, and AC.

When the terms “about” or “substantially” are used in this specification in connection with a numerical value, it is intended that the associated numerical value includes a manufacturing or operational tolerance (e.g., +10%) around the stated numerical value. Moreover, when the words “generally” and “substantially” are used in connection with geometric shapes, it is intended that precision of the geometric shape is not required but that latitude for the shape is within the scope of the disclosure. Further, regardless of whether numerical values or shapes are modified as “about” or “substantially,” it will be understood that these values and shapes should be construed as including a manufacturing or operational tolerance (e.g., +10%) around the stated numerical values or shapes. When ranges are specified, the range includes all values therebetween such as increments of 0.1%.

While the term “equal to” is used in the description of example embodiments, it should be understood that some imprecisions may exist. Thus, when one element is referred to as “equal to” another element, it should be understood that an element or a value may be “equal to” another element within a desired manufacturing or operational tolerance range (e.g., +10%).

The notion that elements are “substantially the same” may indicate that the element may be completely the same and may also indicate that the elements may be determined to be the same in consideration of errors or deviations occurring during a process.

Hereinafter, example embodiments of the present disclosure will be described in detail so that a person skilled in the art would understand the same. However, this disclosure may be embodied in many different forms and is not to be construed as limited to the presented embodiments set forth herein.

In the drawings, the thickness of layers, films, panels, regions, etc., are exaggerated for clarity. It will be understood that when an element such as a layer, film, region, or substrate is referred to as being “on” another element, it may be directly on the other element or intervening elements may also be present. In contrast, when an element is referred to as being “directly on” another element, there are no intervening elements present.

In the drawings, parts having no relationship with the description are omitted for clarity, and the same or similar constituent elements are indicated by the same reference numeral throughout the specification.

Hereinafter, the terms “lower portion” and “upper portion” are for convenience of description and do not limit the positional relationship.

Hereinafter, the term “combination” includes mixed and two or more laminated structures.

As used herein, when a definition is not otherwise provided, “substituted” refers to replacement of a hydrogen atom of a compound by a substituent selected from a halogen, a hydroxy group, a nitro group, a cyano group, an amino group, an azido group, an amidino group, a hydrazino group, a hydrazono group, a carbonyl group, a carbamyl group, a thiol group, an ester group, a carboxyl group or a salt thereof, a sulfonic acid group or a salt thereof, a phosphoric acid group or a salt thereof, a C1 to C30 alkyl group, a C2 to C30 alkenyl group, a C2 to C30 alkynyl group, a C6 to C30 aryl group, a C7 to C30 arylalkyl group, a C1 to C30 alkoxy group, a C1 to C20 heteroalkyl group, a C3 to C20 heterocyclic group, a C3 to C20 heteroarylalkyl group, a C3 to C30 cycloalkyl group, a C3 to C15 cycloalkenyl group, a C6 to C15 cycloalkynyl group, a C3 to C30 heterocycloalkyl group, and a combination thereof.

As used herein, when a definition is not otherwise provided, “hetero” refers to one including 1 to 4 heteroatoms selected from N, O, S, Se, Te, Si, and P.

Hereinafter, a compound according to some example embodiments is described.

A compound according to some example embodiments may be represented by Chemical Formula 1.

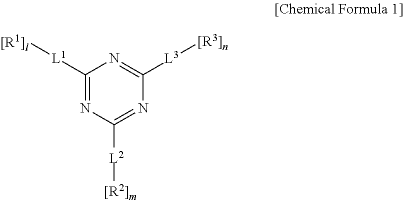

In Chemical Formula 1,
L¹ to L³ each independently may be a single bond, O, S, NR^{a}, a substituted or unsubstituted C1 to C10 oxyalkylene group, a substituted or unsubstituted C1 to C10 thioalkylene group, a substituted or unsubstituted C6 to C30 arylene group, a substituted or unsubstituted divalent C3 to C30 heterocyclic group, or a combination thereof,
R^{a} may be hydrogen, a substituted or unsubstituted C1 to C10 alkyl group, a substituted or unsubstituted C2 to C30 alkenyl group, a substituted or unsubstituted C2 to C30 alkynyl group, a substituted or unsubstituted C1 to C30 alkoxy group, a substituted or unsubstituted C6 to C30 aryl group, a substituted or unsubstituted C3 to C30 heteroaryl group, or a combination thereof,
I, m, and n each independently may be an integer greater than or equal to 1,
R¹ to R³ each independently may be a substituted or unsubstituted C6 to C30 aryl group, a substituted or unsubstituted C3 to C30 heterocyclic group, a substituted or unsubstituted amino group, or a combination thereof,
at least one of R¹ to R³ independently may be a group represented by Chemical Formula A,

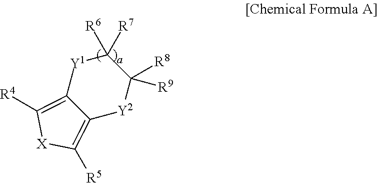

wherein, in Chemical Formula A,
X, Y¹, and Y² each independently may be O, S, Se, or Te,
R⁴ to R⁹ each independently may be hydrogen, a substituted or unsubstituted C1 to C30 alkyl group, a substituted or unsubstituted C2 to C30 alkenyl group, a substituted or unsubstituted C2 to C30 alkynyl group, a substituted or unsubstituted C1 to C30 oxyalkyl group, a substituted or unsubstituted C1 to C30 haloalkyl group, a substituted or unsubstituted C1 to C30 alkoxy group, a halogen, a cyano group, or a linking point with Chemical Formula 1,
a may be an integer from 1 to 3, and
if a is 2 or 3, R⁶ may be the same as or different from each other and R⁷ may be the same as or different from each other.

In Chemical Formula A, at least one of R⁴ to R⁶ may be a linking point with Chemical Formula 1.

The compound may have a structure in which three substituents capable of controlling electrical characteristics of the compound are bonded to a triazine core, and may have an asymmetric structure in which at least one of the three substituents are different from the remainder. At least one of the three substituents may include a reaction site for polymerization in the polymerization process described below.

For example, the compound may include two or more substituents capable of providing different energy bandgaps from each other, and for example, the compound may include three substituents capable of providing different energy bandgaps from each other.

The compound may include an alkylenedioxythiophenyl group represented by Chemical Formula A in at least one of the three substituents, and as described below, a polymer including a structural unit derived from an alkylenedioxythiophenyl group represented by Chemical Formula A may effectively control electrochemical characteristics and optical characteristics, thereby exhibiting improved electrochromic characteristics.

For example, at least one of L¹ to L³ may be different from the remainder of L¹ to L³, or at least one of R¹ to R³ may be different from the remainder of R¹ to R³. Accordingly, the asymmetric structure may be formed around the triazine core.

For example, one or two of L¹ to L³ may each independently be O, S, a substituted or unsubstituted C1 to C10 oxyalkylene group, or a substituted or unsubstituted C1 to C10 thioalkylene group, and the remainder of L¹ to L³ may each independently be NR^{a}.

For example, one or two of L¹ to L³ may each independently be O, S, or NR^{a}, and the remainder of L¹ to L³ may each independently be a substituted or unsubstituted C1 to C10 oxyalkylene group or a substituted or unsubstituted C1 to C10 thioalkylene group.

For example, at least one of R¹ to R³ may be a group represented by Chemical Formula A, and the remainder of R¹ to R³ may each independently be a substituted or unsubstituted phenyl group, a substituted or unsubstituted biphenyl group, a substituted or unsubstituted naphthyl group, a substituted or unsubstituted anthracenyl group, a substituted or unsubstituted fluorenyl group, a substituted or unsubstituted triphenylene group, a substituted or unsubstituted carbazolyl group, a substituted or unsubstituted aniline group, a substituted or unsubstituted pyrrole group, a substituted or unsubstituted indole group, a substituted or unsubstituted thiazole group, a substituted or unsubstituted benzothiazole group, a substituted or unsubstituted thiadiazole group, a substituted or unsubstituted benzothiadiazole group, a substituted or unsubstituted imidazole group, a substituted or unsubstituted benzoimidazole group, a substituted or unsubstituted pyrazolyl group, a substituted or unsubstituted phenylenevinylene group, a substituted or unsubstituted triphenylamine group, or a combination thereof.

For example, any two of R¹ to R³ may be a group represented by Chemical Formula A, and the remainder of R¹ to R³ may be a substituted or unsubstituted phenyl group, a substituted or unsubstituted biphenyl group, a substituted or unsubstituted naphthyl group, a substituted or unsubstituted anthracenyl group, a substituted or unsubstituted fluorenyl group, a substituted or unsubstituted triphenylene group, a substituted or unsubstituted carbazolyl group, a substituted or unsubstituted aniline group, a substituted or unsubstituted pyrrole group, a substituted or unsubstituted indole group, a substituted or unsubstituted thiazole group, a substituted or unsubstituted benzothiazole group, a substituted or unsubstituted thiadiazole group, a substituted or unsubstituted benzothiadiazole group, a substituted or unsubstituted imidazole group, a substituted or unsubstituted benzoimidazole group, a substituted or unsubstituted pyrazolyl group, a substituted or unsubstituted phenylenevinylene group, a substituted or unsubstituted triphenylamine group, or a combination thereof.

For example, I, m, and n may each independently be an integer from 1 to 100.

For example, X may be S or Se, and Y¹ and Y² may each independently be O or S.

For example, X may be different from Y¹ and Y², and Y¹ and Y² may be the same.

For example, X may be S and Y¹ and Y² may each be O.

For example, the group represented by Chemical Formula A may be represented by Chemical Formula A-1 or A-2.

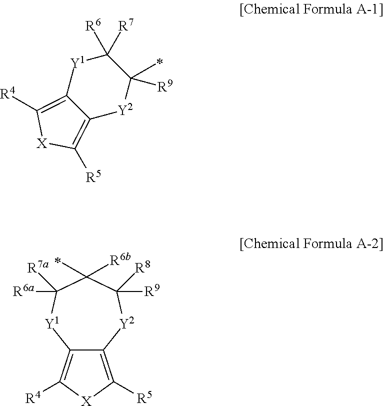

In Chemical Formula A-1 or A-2,
X, Y¹, and Y² each independently may be O, S, Se, or Te,
R⁴, R⁵, R⁶, R^{6a}, R^{6b}, R⁷, R^{7a}, R⁸, and R⁹ each independently may be hydrogen, a substituted or unsubstituted C1 to C30 alkyl group, a substituted or unsubstituted C2 to C30 alkenyl group, a substituted or unsubstituted C2 to C30 alkynyl group, a substituted or unsubstituted C1 to C30 oxyalkyl group, a substituted or unsubstituted C1 to C30 haloalkyl group, a substituted or unsubstituted C1 to C30 alkoxy group, a halogen, a cyano group, or a combination thereof, and
* may be a linking point with Chemical Formula 1.

For example, some of R¹ to R³ may be a group represented by Chemical Formula A-1, and a remainder of R¹ to R³ of Chemical Formula 1 may be a group represented by Chemical Formula A-2.

For example, R¹ to R³ may be different from each other.

For example, R¹ may be a group represented by Chemical Formula A-1, R² may be a group represented by Chemical Formula A-2, and R³ may be a substituted or unsubstituted phenyl group, a substituted or unsubstituted biphenyl group, a substituted or unsubstituted naphthyl group, a substituted or unsubstituted anthracenyl group, a substituted or unsubstituted fluorenyl group, a substituted or unsubstituted triphenylene group, a substituted or unsubstituted carbazolyl group, a substituted or unsubstituted aniline group, a substituted or unsubstituted pyrrole group, a substituted or unsubstituted indole group, a substituted or unsubstituted thiazole group, a substituted or unsubstituted benzothiazole group, a substituted or unsubstituted thiadiazole group, a substituted or unsubstituted benzothiadiazole group, a substituted or unsubstituted imidazole group, a substituted or unsubstituted benzoimidazole group, a substituted or unsubstituted pyrazolyl group, a substituted or unsubstituted phenylenevinylene group, a substituted or unsubstituted triphenylamine group, or a combination thereof.

For example, the compound may be one of those listed in Group 1, but is not limited thereto.

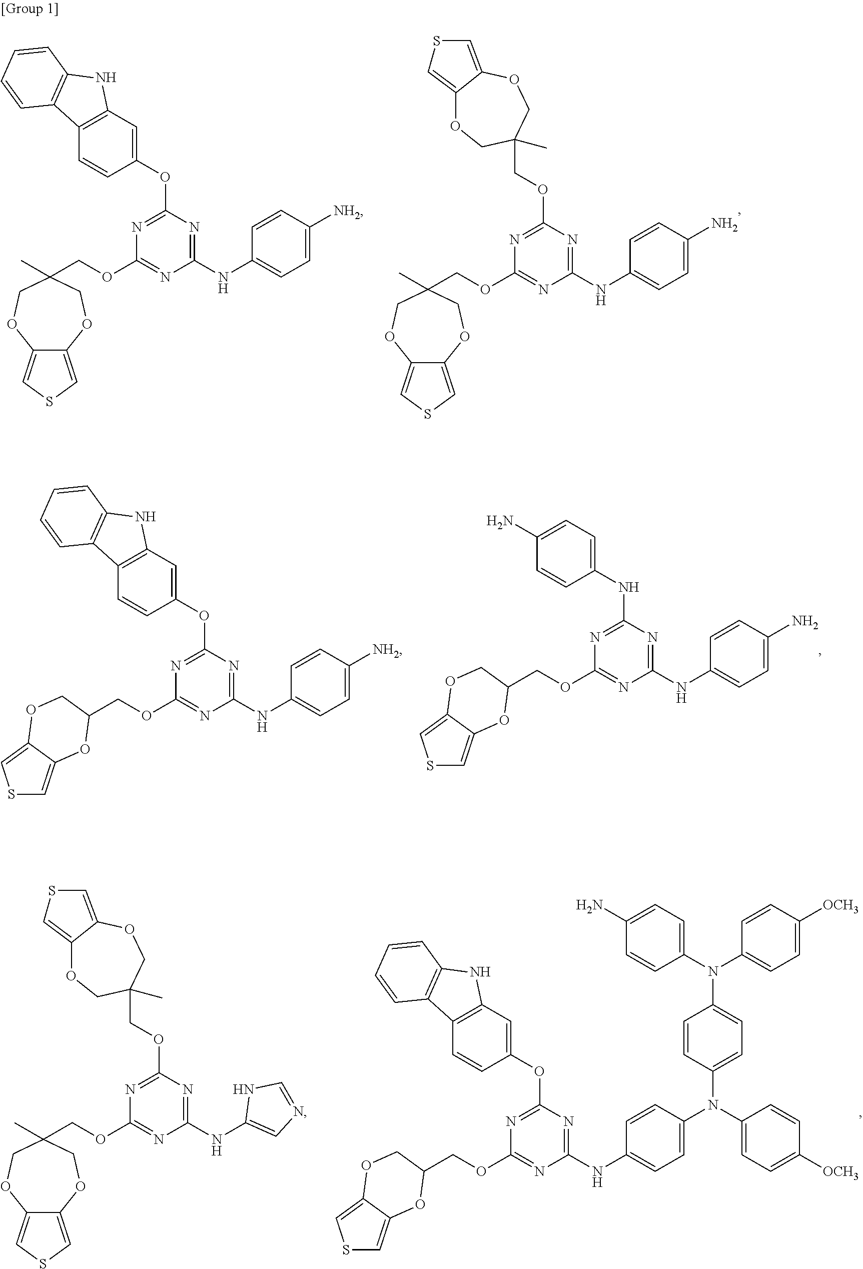 [SMILES: CC1(COc2nc(Nc3ccc(N)cc3)nc(Oc3ccc4c(c3)[nH]c3ccccc34)n2)COc2cscc2OC1 ; CC1(COc2nc(Nc3ccc(N)cc3)nc(OCC3(C)COc4cscc4OC3)n2)COc2cscc2OC1 ; Nc1ccc(Nc2nc(OCC3COc4cscc4O3)nc(Oc3ccc4c(c3)[nH]c3ccccc34)n2)cc1 ; Nc1ccc(Nc2nc(Nc3ccc(N)cc3)nc(OCC3COc4cscc4O3)n2)cc1 ; CC1(COc2nc(Nc3cnc[nH]3)nc(OCC3(C)COc4cscc4OC3)n2)COc2cscc2OC1 ; COc1ccc(N(c2ccc(N)cc2)c2ccc(N(c3ccc(Nc4nc(OCC5COc6cscc6O5)nc(Oc5ccc6c(c5)[nH]c5ccccc56)n4)cc3)c3ccc(OC)cc3)cc2)cc1]

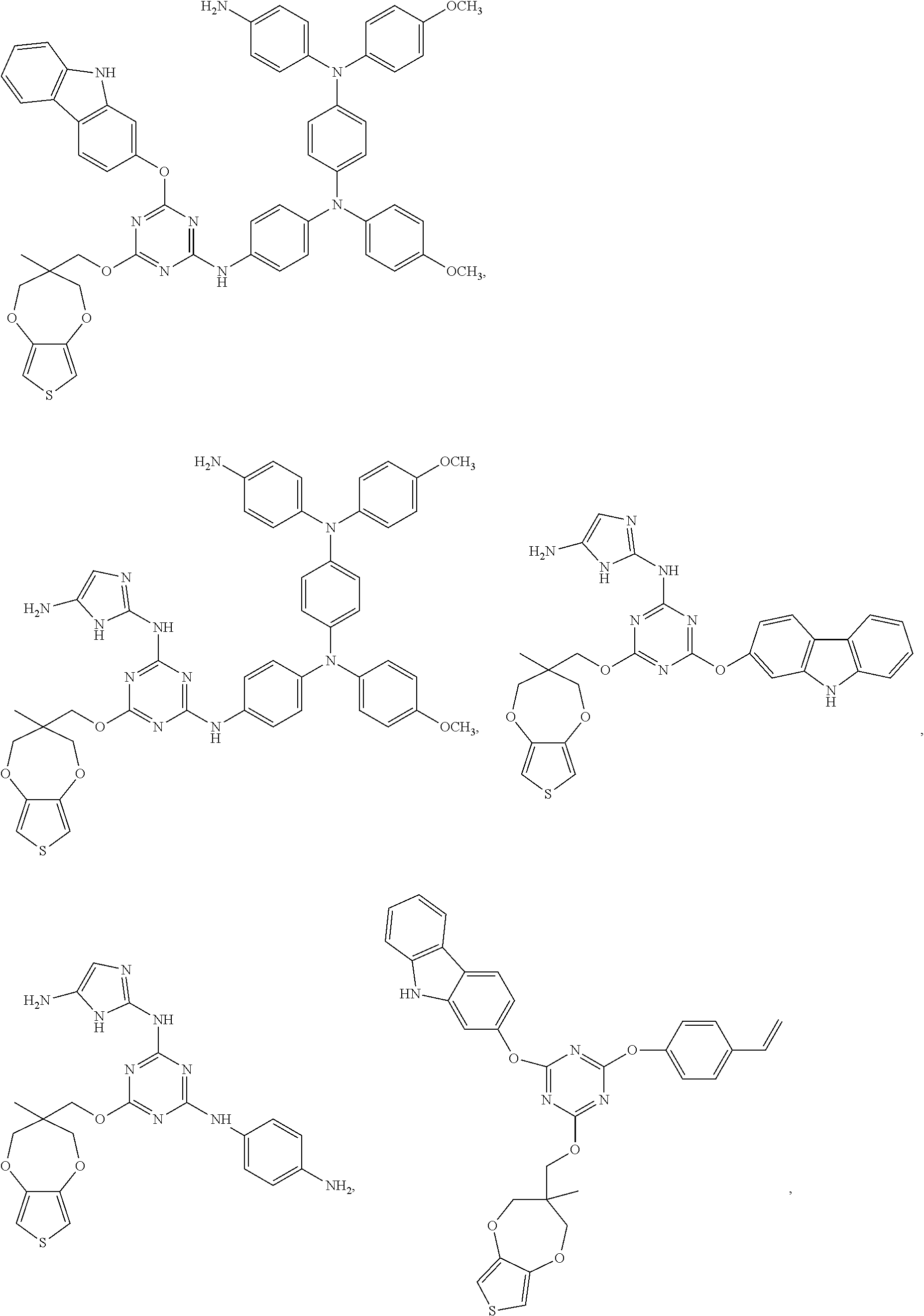 [SMILES: COc1ccc(N(c2ccc(N)cc2)c2ccc(N(c3ccc(Nc4nc(OCC5(C)COc6cscc6OC5)nc(Oc5ccc6c(c5)[nH]c5ccccc56)n4)cc3)c3ccc(OC)cc3)cc2)cc1 ; COc1ccc(N(c2ccc(N)cc2)c2ccc(N(c3ccc(Nc4nc(Nc5ncc(N)[nH]5)nc(OCC5(C)COc6cscc6OC5)n4)cc3)c3ccc(OC)cc3)cc2)cc1 ; CC1(COc2nc(Nc3ncc(N)[nH]3)nc(Oc3ccc4c(c3)[nH]c3ccccc34)n2)COc2cscc2OC1 ; CC1(COc2nc(Nc3ccc(N)cc3)nc(Nc3ncc(N)[nH]3)n2)COc2cscc2OC1 ; C=Cc1ccc(Oc2nc(OCC3(C)COc4cscc4OC3)nc(Oc3ccc4c(c3)[nH]c3ccccc34)n2)cc1]

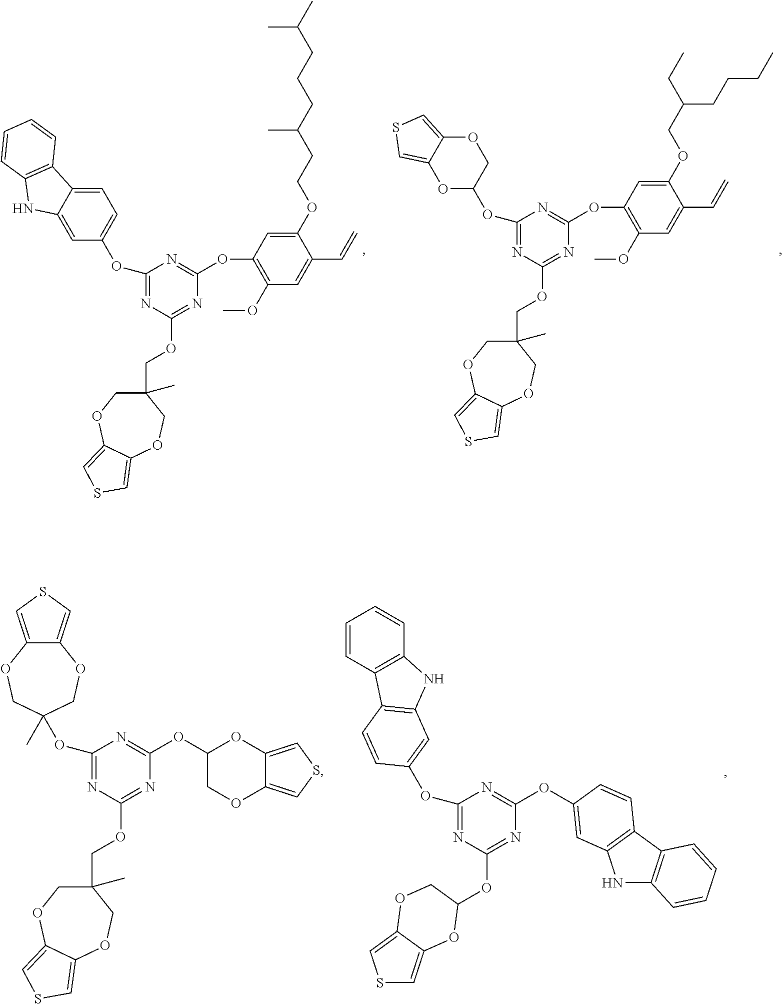 [SMILES: C=Cc1cc(OC)c(Oc2nc(OCC3(C)COc4cscc4OC3)nc(Oc3ccc4c(c3)[nH]c3ccccc34)n2)cc1OCCC(C)CCCC(C)C ; C=Cc1cc(OC)c(Oc2nc(OCC3(C)COc4cscc4OC3)nc(OC3COc4cscc4O3)n2)cc1OCC(CC)CCCC ; CC1(COc2nc(OC3COc4cscc4O3)nc(OC3(C)COc4cscc4OC3)n2)COc2cscc2OC1 ; c1ccc2c(c1)[nH]c1cc(Oc3nc(Oc4ccc5c(c4)[nH]c4ccccc45)nc(OC4COc5cscc5O4)n3)ccc12]

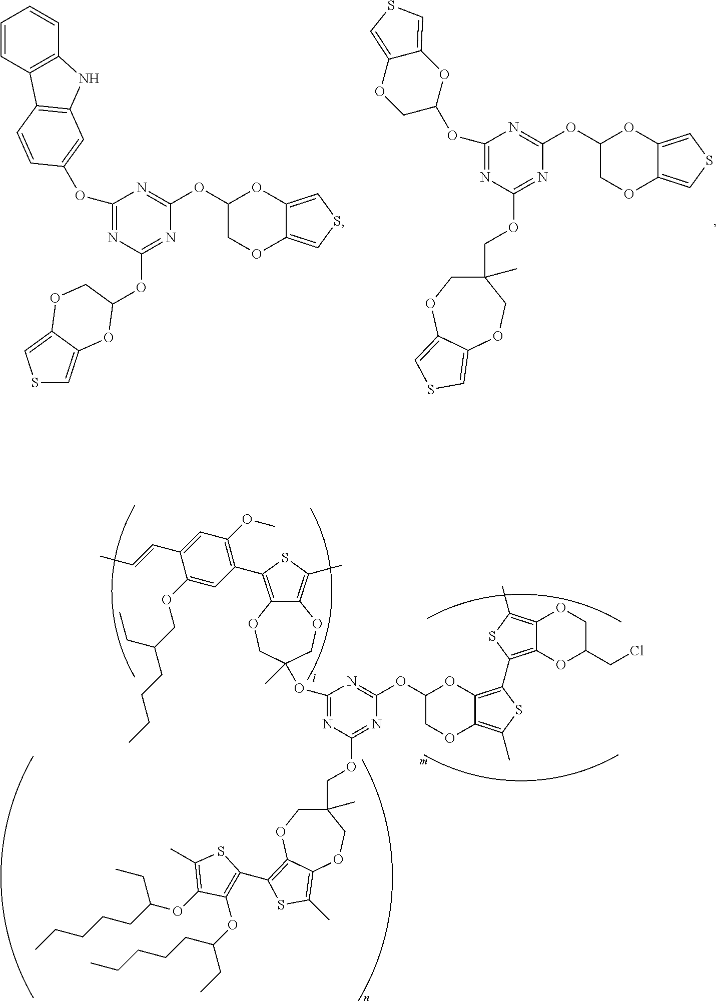

The aforementioned compound may be polymerized alone or with other types of compounds to form a polymer.

The polymer may include a structural unit derived from the aforementioned compound.

As described above, the polymer derived from the compound including the triazine core and at least one alkylenedioxythiophenyl group represented by Chemical Formula A may effectively control (and/or improve control of) electrochemical and optical characteristics, and thus may serve as an improved electrochromic material.

Specifically, the polymer may effectively control (and/or improve control of) an electrochemical potential and/or the light absorbency in the ultraviolet, visible and/or infrared regions, and may realize, for example, blackness that limits and/or blocks all wavelengths of visible light at a first voltage and transparency that transmits all wavelengths of visible light at a second voltage. Accordingly, it may be configured to reversibly display black or transparency depending on the applied voltage, and for example, a difference in light transmittance depending on the applied voltage for light of a wavelength of about 500 nm may be greater than or equal to about 60%, and within the above range may be greater than or equal to about 70%, greater than or equal to about 75%, greater than or equal to about 80%, greater than or equal to about 85%, greater than or equal to about 90%, or greater than or equal to about 95%.

For example, the polymer may include at least one of R¹, R², or R³ of Chemical Formula 1 as a repeating unit.

For example, the polymer may include a repeating unit having R⁴ and/or R⁵ of Chemical Formula A as a linking group.

For example, the polymer may further include a structural unit derived from the compound having a higher degree of polymerization. For example, the polymer may further include a moiety derived from polyalkylene glycol, for example, a moiety derived from polyethylene glycol, polypropylene glycol, or a combination thereof.

For example, the polymer may be obtained in a film forming process, for example, by coating and polymerizing a solution including one or more compounds. For example, the film formation may include coating a mixture of a solution including the aforementioned compound represented by Chemical Formula 1 and a solution including a compound having a higher degree of polymerization, and polymerizing by heat treatment. A film (polymer film) including such a polymer may exhibit higher film stability and/or uniformity.

For example, the polymer film may effectively control (and/or provide improved control of) the electrochemical potential and/or the optical absorbance in the ultraviolet, visible and/or infrared regions depending on the applied voltage, and may, for example, realize black that limits and/or blocks all wavelengths of visible light at a first voltage and transparent that transmits all wavelengths of visible light at a second voltage. Accordingly, the polymer film may be configured to reversibly display black or transparency depending on the applied voltage, and for example, the difference in light transmittance depending on the applied voltage for light of a wavelength of about 500 nm may be greater than or equal to about 60%, and within the above range may be greater than or equal to about 70%, greater than or equal to about 75%, greater than or equal to about 80%, greater than or equal to about 85%, greater than or equal to about 90%, or greater than or equal to about 95%.

The aforementioned polymer or polymer film may be applied to various devices and electronic devices requiring electrochemical characteristics.

As an example, the aforementioned polymer may be applied as an electrochromic material in an electrochromic device requiring electrochemical and optical characteristics.

An electrochromic device according to some example embodiments is described below.

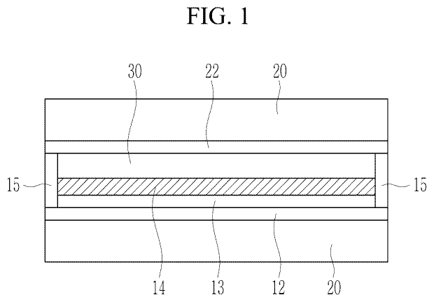

FIG. 1 is a cross-sectional view illustrating an example of an electrochromic device according to some example embodiments.

Referring to FIG. 1, an electrochromic device according to some example embodiments includes a lower substrate 10 and an upper substrate 20 facing each other and fixed by a spacer 15, a lower electrode 12 and an upper electrode 22 on one surface of each of the lower substrate 10 and the upper substrate 20, an electrochromic layer 14 between the lower electrode 12 and the upper electrode 22, and an electrolyte 30 in contact with the electrochromic layer 14 between the lower electrode 12 and the upper electrode 22.

The lower substrate 10 and the upper substrate 20 may be made of transparent glass or a polymer, and the polymer may include one or more selected from, for example, polyacrylate, polyethylene ether phthalate, polyethylene naphthalate, polycarbonate, polyarylate, polyether imide, polyether sulfone, and polyimide.

At least one of the lower electrode 12 or the upper electrode 22 may be made of a transparent conductor, and each of the lower electrode 12 and the upper electrode 22 may include an inorganic conductive material such as indium tin oxide (ITO), fluorine tin oxide (FTO), or antimony doped tin oxide (ATO), or an organic conductive material such as polyacetylene or polythiophene.

The electrochromic layer 14 includes an electrochromic material. The electrochromic material may be a compound configured to reversibly display color or change color by electrochemical change depending on the direction of an electric field when voltage is applied, and may include the aforementioned polymer. For example, the electrochromic layer 14 may be the polymer film described above. For example, the polymer may include one or more types, and may include a first polymer and a second polymer including different repeating units.

The electrochromic material may be cathodic coloration materials or anodic coloration materials. The cathodic coloration material may be a substance that displays a color (including black) in a reduced state and becomes transparent in an oxidized state, and the anodic coloration material may be a substance that displays a color (including black) in an oxidized state and becomes transparent in a reduced state.

For example, the electrochromic material may be adsorbed on nanoparticles such as titanium dioxide (TiO₂). At this time, the nanoparticles may be semiconductor materials, and for example, titanium oxide (TiO₂), zinc oxide (ZnO₂), tungsten oxide (WO₃), or a combination thereof may be used. The shape of the nanoparticles may be various, such as sphere, tetrahedron, cylinder, triangle, disk, tripod, tetrapod, cube, box, star, tube, etc., and the size of the nanoparticles may be about 1 to about 100 nm.

The electrochromic layer 14 may have one layer or two or more layers. For example, the electrochromic layer 14 may include a first electrochromic layer (not shown) and a second electrochromic layer (not shown) including polymers having different repeating units. The first electrochromic layer and the second electrochromic layer may have different optical characteristics from each other, and the combination of the first electrochromic layer and the second electrochromic layer may increase the difference in light transmittance described above, thereby reversibly displaying black or transparency more effectively.

The auxiliary layer 13 may be included between the lower electrode 12 and the electrochromic layer 14 to improve the adhesion between the lower electrode 12 and the electrochromic layer 14. However, the auxiliary layer 13 may be omitted.

The electrolyte 30 may be filled between the lower electrode 12 and the upper electrode 22 and may be in contact with the electrochromic layer 14.

The electrolyte 30 may supply a substance that promotes an oxidation/reduction reaction of the electrochromic material and may include a liquid electrolyte or a solid polymer electrolyte. The liquid electrolyte or solid polymer electrolyte may be an ionic substance, and as the ionic liquid electrolyte, a solution in which lithium salts such as LiOH or LiClO₄, potassium salts such as KOH, and sodium salts such as NaOH are dissolved in a solvent may be used, but is not limited thereto. Examples of the ionic solid electrolyte that may be used may include, but are not limited to, poly(2-acrylamino-2-methylpropane sulfonic acid), poly(ethylene oxide), 1-butyl-3-methylimidazolium bis(trifluoromethylsulfonyl)imide (BMIMTFSI), and/or 1-butyl-3-methylimidazolium tetrafluoroborate (BMIMMBF4).

The aforementioned electrochromic device may be applied to various electronic devices that may require control of the inflow of light through color control, such as optical filters, electronic signboards, smart windows, or wearable imaging devices (such as AR or VR).

Hereinafter, some example embodiments are illustrated in more detail with reference to examples. However, these examples are non-limiting, and the present scope is not limited thereto.

### SYNTHESIS EXAMPLES

### Synthesis Example 1

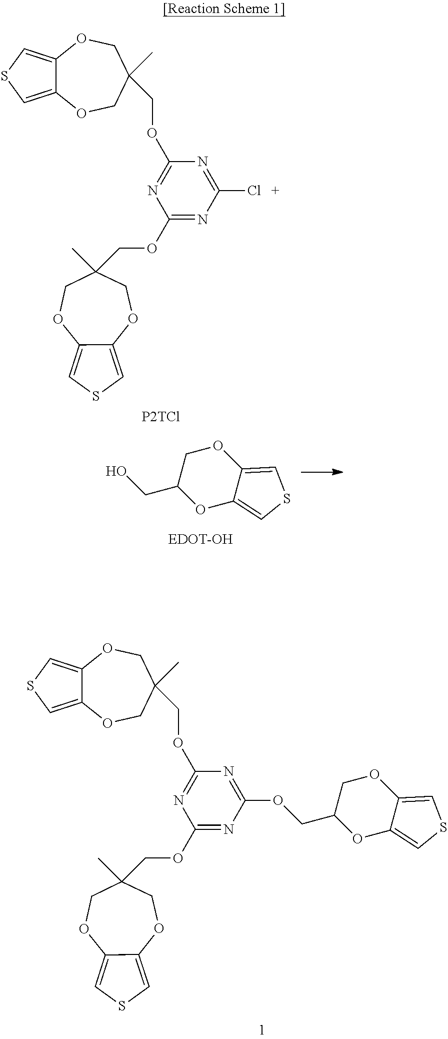

### Synthesis of P2TCI

In a two-necked 100 ml flask equipped with a reflux device, triazine-CI3 (2,4,6-trichloro-1,3,5-triazine, 5 mmol) is dissolved in a mixed solution of acetone/deionized water (in a volume ratio of 4/1). Subsequently, 10.1 mmol (2 eq) of a hydroxy-substituted 3,4-propylenedioxythiophene (hydroxy-ProDOT) and 5 mmol (1 eq) of sodium hydroxide are added thereto and then, reacted at 0° C. for 4 hours. After the reaction, when a white precipitate occurs, deionized water at 0° C. is added thereto to terminate the reaction. Subsequently, after passing a solution including the precipitate through an apparatus for the filtration under reduced pressure, the white solid on a filter paper is twice washed with deionized water and dried to obtain 2,4-bis((3,4-dihydro-3-methyl-2H-thieno[3,4-b][1,4]dioxepin-3-yl) methoxy)-6-chloro-1,3,5-triazine (P2TCI) (Yield: 90%).

¹H NMR (300 MHZ, Chloroform-d, o): 5.9 (s, 4H), 4.41-4.16 (m, 4H), 3.98-3.73 (m, 6H), 1.53 (s, 3H), 1.16 (s, 3H).

### Synthesis of Compound 1

In a two-necked 100 ml flask equipped with a reflux device, 153 mg of P2TCI is dissolved in 10 ml of tetrahydrofuran anhydride. While operating the reflux device, 198 mg of EDOT-OH (2,3-dihydrothieno[3,4-b][1,4]dioxin-2-yl) methanol) and 40 mg of sodium hydride are dissolved in 2 ml of tetrahydrofuran anhydride and then, reacted at 70° C. for 2 days. After the reaction, the resultant is cooled to room temperature, and 20 ml of deionized water is added thereto to terminate the reaction. Subsequently, the solution including a precipitate is passed through a vacuum filtration device, and the solid on a filter paper is twice washed with deionized water to obtain Compound 1 (2-((2,3-dihydrothieno[3,4-b][1,4]dioxin-2-yl) methoxy)-4,6-bis((3,4-dihydro-3-methyl-2H-thieno[3,4-b][1,4]dioxepin-3-yl) methoxy)-1,3,5-triazine).

¹H NMR (300 MHZ, Chloroform-d, o): 6.51 (m, 6H), 6.47 (d, 1H), 4.7 (m, 2H), 4.45-4.20 (m, 4H), 3.98-3.73 (m, 6H), 1.53 (s, 3H), 1.16 (s, 3H).

### Synthesis Example 2

### Synthesis of Intermediate I-1

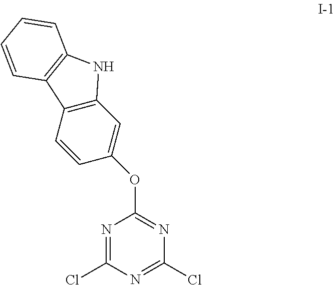 [SMILES: Clc1nc(Cl)nc(Oc2ccc3c(c2)[nH]c2ccccc23)n1]

In a two-necked 100 ml flask equipped with a reflux device, triazine (5 mmol) is dissolved in a mixed solution of acetone/deionized water (in a volume ratio of 4/1). Subsequently, 5 mmol (1 eq) of hydroxy-carbazole and 5 mmol (1 eq) of sodium hydroxide are added thereto and then, reacted at 0° C. for 4 hours. After the reaction, when a white precipitate occurs, deionized water at 0° C. is added thereto to terminate a reaction. Subsequently, a solution including the precipitate is passed through an apparatus for filtration under reduced pressure, and the white solid on a filter pater is twice washed with deionized water to obtain Intermediate I-1 (2-(4,6-dichloro-1,3,5-triazin-2-yloxy)-9H-carbazole).

1H NMR (300 MHZ, Chloroform-d, o): 7.55-7.38 (m, 3H), 7.24 (s, 1H), 7.08-7.00 (m, 2H), 6.87 (s, 1H), 6.47 (d, 1H).

### Synthesis of Intermediate I-2

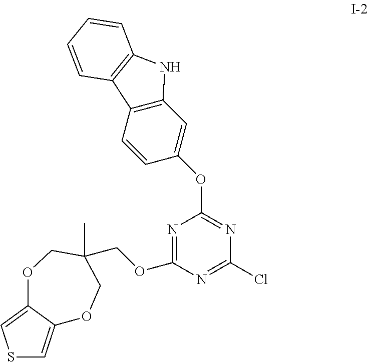 [SMILES: CC1(COc2nc(Cl)nc(Oc3ccc4c(c3)[nH]c3ccccc34)n2)COc2cscc2OC1]

In a two-necked 100 ml flask equipped with a reflux device, Intermediate I-1 (5 mmol) is dissolved in a mixed solution of acetone/deionized water (in a volume ratio of 4/1). Subsequently, 5 mmol (1 eq) of hydroxy-ProDOT and 5 mmol (1 eq) of sodium hydroxide are added thereto and then, reacted at 0° C. for 1 hour. The reaction proceeds at room temperature for 48 hours to obtain a white precipitate. After the reaction, the resultant is cooled to room temperature, and deionized water is added thereto to terminate the reaction. Subsequently, a solution including the precipitate is passed through a vacuum filtration device, and the solid on a filter pater is twice washed with deionized water to obtain Intermediate I-2 (2-(4-((3,4-dihydro-3-methyl-2H-thieno[3,4-b][1,4]dioxepin-3-yl) methoxy)-6-chloro-1,3,5-triazin-2-yloxy)-9H-carbazole).

1H NMR (300 MHZ, Chloroform-d, δ): 7.55-7.38 (m, 3H), 7.24 (s, 1H), 7.08-7.00 (m, 2H), 6.87 (s, 1H), 6.47 (d, 1H), 5.9 (s, 2H), 3.98-3.73 (m, 6H), 1.16 (s, 3H).

### Synthesis of Compound 2

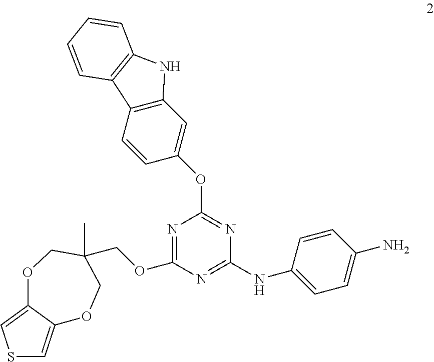 [SMILES: CC1(COc2nc(Nc3ccc(N)cc3)nc(Oc3ccc4c(c3)[nH]c3ccccc34)n2)COc2cscc2OC1]

In a two-necked 100 ml flask equipped with a reflux device, Intermediate I-2 is dissolved in a mixed solution of acetone/deionized water (in a volume ratio of 4/1). Subsequently, 5 mmol (1 eq) of para-phenylenediamine and 5 mmol (1 eq) of potassium bicarbonate is dissolved in a small amount of THE solution in a 4 ml vial and then, added to the reaction solution, which is reacted at 70° C. for 4 days. After the reaction, the resultant is cooled to room temperature, and deionized water is added thereto to terminate the reaction. Subsequently, a solution including a precipitate is passed through an apparatus for filtration under reduced pressure, and the solid on a filter pater is twice washed with deionized water to obtain Compound 2 (N1-(4-((3,4-dihydro-3-methyl-2H-thieno[3,4-b][1,4]dioxepin-3-yl) methoxy)-6-(9H-carbazol-2-yloxy)-1,3,5-triazin-2-yl)benzene-1,4-diamine).

¹H NMR (300 MHZ, Chloroform-d, 0): 7.55-7.38 (m, 3H), 7.24 (s, 1H), 7.08-7.00 (m, 2H), 6.87 (s, 1H), 6.47 (d, 1H), 6.21 (d, 4H), 5.9 (s, 2H), 5.01-4.99 (m, 3H), 3.98-3.73 (m, 6H), 1.16 (s, 3H).

### Synthesis Example 3

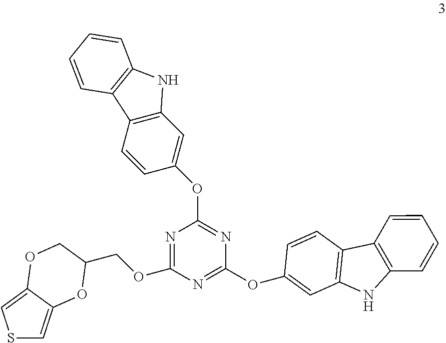 [SMILES: c1ccc2c(c1)[nH]c1cc(Oc3nc(OCC4COc5cscc5O4)nc(Oc4ccc5c(c4)[nH]c4ccccc45)n3)ccc12]

In a two-necked 100 ml flask equipped with a reflux device, 2-(4-((3,4-dihydro-3-methyl-2H-thieno[3,4-b][1,4]dioxepin-3-yl) methoxy)-6-chloro-1,3,5-triazin-2-yloxy)-9H-carbazole is dissolved in a mixed solution of acetone/deionized water (in a volume ratio of 4/1). While operating the reflux device, 5 mmol (1 eq) of hydroxy-EDOT and 5 mmol (1 eq) of sodium hydroxide are added to the reaction solution and then, reacted at 70° C. for 4 days. After the reaction, the resultant is cooled to room temperature, and deionized water is added thereto to terminate the reaction. Subsequently, the solution including a precipitate is passed through an apparatus for filtration under reduced pressure, and the light gray solid on a filter pater is twice washed with deionized water to obtain Compound 3 (2-(4-((2,3-dihydrothieno[3,4-b][1,4]dioxin-2-yl) methoxy)-6-(9H-carbazol-2-yloxy)-1,3,5-triazin-2-yloxy)-9H-carbazole).

¹H NMR (300 MHZ, Chloroform-d, δ): 7.55-7.38 (m, 6H), 7.24 (s, 2H), 7.08-7.00 (m, 4H), 6.87 (s, 2H), 6.47 (d, 2H), 5.9 (s, 2H), 4.7 (m, 1H), 4.45-4.20 (m, 4H).

### Synthesis Example 4

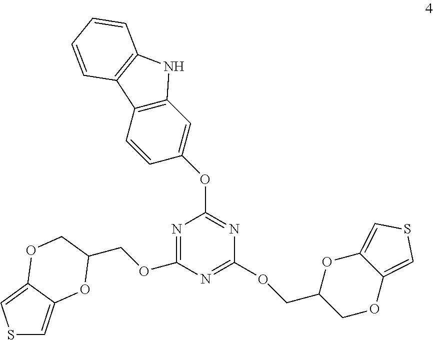 [SMILES: c1ccc2c(c1)[nH]c1cc(Oc3nc(OCC4COc5cscc5O4)nc(OCC4COc5cscc5O4)n3)ccc12]

In a two-necked 100 ml flask equipped with a reflux device, the 2-(4-((3,4-dihydro-3-methyl-2H-thieno[3,4-b][1,4]dioxepin-3-yl) methoxy)-6-chloro-1,3,5-triazin-2-yloxy)-9H-carbazole is dissolved in a mixed solution of acetone/deionized water (in a volume ratio of 4/1). While operating the reflux device, 5 mmol (1 eq) of hydroxy-EDOT and 5 mmol (1 eq) of sodium hydroxide are added thereto and then, reacted at 70° C. for 4 days. After the reaction, deionized water is added thereto at room temperature to terminate the reaction. Subsequently, the solution including a precipitate is passed through an apparatus for filtration under reduced pressure, and the light gray solid on a filter paper is twice washed with deionized water to obtain Compound 4 (2-(4,6-bis((2,3-dihydrothieno[3,4-b][1,4]dioxin-2-yl) methoxy)-1,3,5-triazin-2-yloxy)-9H-carbazole).

¹H NMR (300 MHZ, Chloroform-d, o): 7.55-7.38 (m, 3H), 7.24 (s, 1H), 7.08-7.00 (m, 2H), 6.87 (s, 1H), 6.47 (d, 1H), 5.9 (s, 4H), 4.7 (m, 2H), 4.45-4.20 (m, 8H).

### Synthesis Example 5

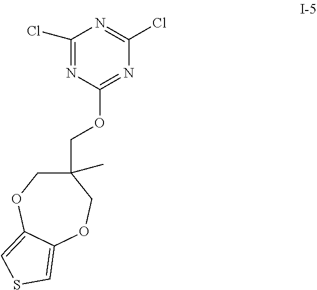 [SMILES: CC1(COc2nc(Cl)nc(Cl)n2)COc2cscc2OC1]

### Synthesis of Intermediate I-5

In a two-necked 100 ml flask equipped with a reflux device, triazine-CI3 (2,4,6-trichloro-1,3,5-triazine, 5 mmol) is dissolved in a mixed solution of acetone/deionized water (in a volume ratio of 4/1). Subsequently, 5.1 mmol (1 eq) of hydroxy-ProDOT and 5 mmol (1 eq) of sodium hydroxide are added thereto and then, reacted at −2° C. for 5 hours. After the reaction, when a white precipitate occurs, deionized water at 0° C. is added thereto to terminate the reaction. Subsequently, the solution including the precipitate is passed through an apparatus for filtration under reduced pressure, and the white solid on a filter paper is twice washed with deionized water and dried to obtain Intermediate I-5 (Yield: 85%).

¹H NMR (300 MHZ, Chloroform-d, o): 5.9 (s, 4H), 4.43-4.23 (m, 4H), 3.98-3.80 (m, 6H), 1.55 (s, 3H), 1.18 (s, 3H).

### Synthesis of Compound 5

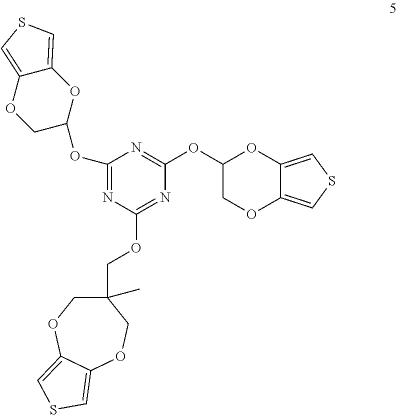 [SMILES: CC1(COc2nc(OC3COc4cscc4O3)nc(OC3COc4cscc4O3)n2)COc2cscc2OC1]

In a two-necked 100 ml flask equipped with a reflux device, 170 mg of Intermediate I-5 is dissolved in 15 ml of methoxy tetrahydrofuran anhydride. While operating the reflux device, 400 mg of EDOT-OH (2,3-dihydrothieno[3,4-b][1,4]dioxin-2-yl) methanol) and 40 mg of sodium hydride are dissolved in 3 ml of tetrahydrofuran anhydride and then, reacted at 80° C. for 52 hours. After the reaction, the resultant is cooled to room temperature, and 20 ml of deionized water is added thereto to terminate the reaction. Subsequently, the solution including a precipitate is passed through an apparatus for filtration under reduced pressure, and the solid on a filter paper is three times washed deionized water to obtain Compound 5 (Yield: 70%).

¹H NMR (300 MHZ, Chloroform-d, δ): 6.51 (m, 6H), 6.50-6.43 (t-m, 2H), 4.7-4.55 (m, 4H), 3.98-3.73 (m, 6H), 1.26 (s, 3H).

### PREPARATION EXAMPLES

### Preparation Example 1

A glass plate coated with FTO (fluorine-doped tin oxide) is washed by using an ultrasonic cleaner with a solvent such as water, acetone, and ethanol and dried at 70° C. in an oven to prepare a transparent electrode.

9.3 mg of PECI (2-chloromethyl-2,3-dihydrothieno[3,4-b]-1,4-dioxine), 17 mg of EHO (3,4-bis(octan-3-yloxy)thiophene) and 7 mg of Compound 1 according to Synthesis Example 1 are dissolved in 100 μl of THF to prepare Solution A, and 0.25 g of FeCl₃·6H₂O and 0.2 g of PEPG (poly(ethylene glycol)-block-poly(propylene glycol)-block-poly(ethylene glycol)) are dissolved in 0.72 g of 1-butanol to prepare Solution B. Subsequently, a mixed solution of Solution A and Solution B is spin-coated on the prepared transparent electrode at a rotational speed of 1500 rpm for 30 seconds and polymerized at 70° C. on a hot plate for 1 hour to obtain a polymer. Then, the polymer is washed with ethanol and dried to form a 200 nm-thick electrochromic layer and thereby, manufacturing a transparent electrode/electrochromic layer stack.

It is confirmed that the electrochromic layer has a uniform surface, and when stored at room temperature, the film uniformity is maintained and stable.

### Preparation Example 2

A glass plate coated with FTO (fluorine-doped tin oxide) is washed by using an ultrasonic cleaner with a solvent such as water, acetone, and ethanol and dried at 70° C. in an oven to prepare a transparent electrode.

9.3 mg of PECI (2-chloromethyl-2,3-dihydrothieno[3,4-b]-1,4-dioxine), 8.2 mg of carbazole, 17 mg of EHO (3,4-bis(octan-3-yloxy)thiophene), and 7 mg of the compound of Synthesis Example 1 are dissolved in 100 μl of THE to prepare Solution A, and 0.25 g of FeCl₃·6H₂O and 0.2 g of PEPG (poly(ethylene glycol)-block-poly(propylene glycol)-block-poly(ethylene glycol)) are dissolved in 0.72 g of 1-butanol to prepare Solution B. Subsequently, a mixed solution of Solution A and Solution B is spin-coated on the prepared transparent electrode at a rotational speed of 1500 rpm for 30 seconds and polymerized at 70° C. on a hot plate for 1 hour to obtain a polymer. Subsequently, the polymer is washed with ethanol and dried to form a 200 nm-thick electrochromic layer and thereby manufacturing a transparent electrode/electrochromic layer stack.

### Preparation Example 3

A glass plate coated with FTO (fluorine-doped tin oxide) is washed by using an ultrasonic cleaner with a solvent such as water, acetone, and ethanol and dried at 70° C. in an oven to prepare a transparent electrode.

9.3 mg of PECI (2-chloromethyl-2,3-dihydrothieno[3,4-b]-1,4-dioxine), 17 mg of EHO (3,4-bis(octan-3-yloxy)thiophene), and 7 mg of Compound 1 of Synthesis Example 1 are dissolved in 100 μl of THE to prepare Solution A and 0.25 g of FeCl₃·6H₂O and 0.2 g of PEPG (poly(ethylene glycol)-block-poly(propylene glycol)-block-poly(ethylene glycol)) are dissolved in 0.72 g of 1-butanol to prepare Solution B. Subsequently, a mixed solution of Solution A and Solution B is spin-coated on the prepared transparent electrode at a rotational speed of 1500 rpm for 30 seconds, and then, a solution prepared by dissolving 10 mg of poly(2-methoxy-5-(3′,7′-dimethyloctyloxy)-1,4-phenylenevinylene) in 300 μl of THE is spin-coated thereon at a rate of 1000 rpm for 30 seconds and polymerized at 70° C. on a hot plate for 1 hour to obtain a polymer. Subsequently, the polymer is washed with ethanol and dried to form a 200 nm-thick electrochromic layer and thereby manufacturing a transparent electrode/electrochromic layer stack.

### Preparation Example 4

A glass plate coated with FTO (fluorine-doped tin oxide) is washed by using an ultrasonic cleaner with a solvent such as water, acetone, and ethanol and dried at 70° C. in an oven to prepare a transparent electrode.

9.3 mg of PECI (2-chloromethyl-2,3-dihydrothieno[3,4-b]-1,4-dioxine), 17 mg of EHO (3,4-bis(octan-3-yloxy)thiophene), 7 mg of the compound according to Synthesis Example 1, and 7 mg of 2-methoxy-5-(3′,7′-dimethyloctyloxy)-1,4-phenylenevinylene are dissolved in 100 μl of THE to prepare Solution A, and 0.25 g of FeCl₃·6H₂O and 0.2 g of PEPG (poly(ethylene glycol)-block-poly(propylene glycol)-block-poly(ethylene glycol)) are dissolved in 0.72 g of 1-butanol to prepare Solution B. Subsequently, a mixed solution of Solution A and Solution B is spin-coated on the prepared transparent electrode at a rotational speed of 1500 rpm for 30 seconds and polymerized at 70° C. on a hot plate for 1 hour to obtain a polymer. Then, the polymer is washed with ethanol and dried to form a 200 nm-thick electrochromic layer and thereby, manufacturing a transparent electrode/electrochromic layer stack.

### Comparative Preparation Example 1

A transparent electrode/electrochromic layer stack is formed in the same manner as in Preparation Example 1 except that Solution A not including Compound 1 of Synthesis Example 1 is used.

The formed electrochromic layer exhibits a non-uniform and crystalline surface.

### EXAMPLES

### Example 1

A glass plate coated with FTO (fluorine-doped tin oxide) is washed by using an ultrasonic cleaner with a solvent such as water, acetone, and ethanol solvent and dried at 70° C. in an oven to prepare a transparent electrode. Subsequently, a TiO₂ dispersion, which is obtained by diluting a TiO₂ nanoparticle paste in ethanol at a concentration of 20 wt %, is spin-coated on the transparent electrode at a rotational speed of 1500 rpm for 30 seconds and dried at 500° C. for 1 hour to form a 600 nm-thick TiO₂ electroactive layer.

Subsequently, onto the electrochromic layer of the transparent electrode/electrochromic layer stack of Preparation Example 1, BMIMTFSI (1-butyl-3-methylimidazolium bis(trifluoromethylsulfonyl)imide) ion gel including 25 wt % of PVDF-HFP (M.W about 100,000) is cut and attached without forming air bubbles to form an electrolyte layer.

Then, the electrolyte layer is covered with the TiO₂ electroactive layer in a sandwich form to manufacture an electrochromic device with an active area of about 1×1.5 cm².

### Example 2

An electrochromic device is manufactured in the same manner as in Example 1 except that the transparent electrode/electrochromic layer stack of Preparation Example 2 is used instead of the transparent electrode/electrochromic layer stack of Preparation Example 1.

### Example 3

An electrochromic device is manufactured in the same manner as in Example 1 except that the transparent electrode/electrochromic layer stack of Preparation Example 3 is used instead of the transparent electrode/electrochromic layer stack of Preparation Example 1.

### Example 4

An electrochromic device is manufactured in the same manner as in Example 1 except that the transparent electrode/electrochromic layer stack of Preparation Example 4 is used instead of the transparent electrode/electrochromic layer stack of Preparation Example 1.

### Example 5

An electrochromic device is manufactured in the same manner as in Example 1 except that the first electrochromic layer is formed by using PRBr (ProDOT-Br2: 3,3-bis(bromomethyl)-3,4-dihydro-2H-thieno-[3,4-b][1,4]dioxepine)) instead of PECI and EHO and Compound 5 of Synthesis Example 5 instead of the compound of Synthesis Example 1 in Preparation Example 1, and the second electrochromic layer is formed by using CoPW@NiO.

Specifically, 30 mg of PRBr (3,3-bis(bromomethyl)-3,4-dihydro-2H-thieno-[3,4-b][1,4]dioxepine)) and Compound 5 of Synthesis Example 5 are dissolved in 100 μl of THE to prepare Solution A, and 0.30 g of FeCl₃·6H₂O and 0.25 g of PEPG (poly(ethylene glycol)-block-poly(propylene glycol)-block-poly(ethylene glycol)) are dissolved in 0.80 g of 2-butanol to prepare Solution B. Subsequently, a mixed solution of Solution A and Solution B is spin-coated on the transparent electrode at a rotational speed of 2000 rpm for 40 seconds and then, polymerized at 75° C. on a hot plate for 1.5 hours to a polymer. Then, the polymer is washed with ethanol and dried to form a 200 nm-thick electrochromic layer and thereby, manufacturing a transparent electrode/first electrochromic layer stack.

A glass electrode coated with FTO (fluorine-doped tin oxide) is washed by using an ultrasonic cleaner with a solvent such as water, acetone, and ethanol and dried at 70° C. in an oven to prepare a transparent electrode, and a nickel oxide dispersion is spin-coated at a rotational speed of 1500 rpm for 30 seconds and heated at 300° C. for 1 hour to form a NiOx film on the FTO glass electrode. The nickel oxide film is immersed in a 0.2 molar citric acid aqueous solution for one day, washed with deionized water, and after dissolving [Co₄(H₂O)₂(PW₉O₃₄)₂]₁₀_{^{−}} substituted with TBA cations in acetonitrile at a concentration of 0.1 mg/ml, the nickel oxide film is placed in a sealed state for one day. Subsequently, the nickel oxide film is washed with distilled water and dried with N2 gas to prepare a second electroactive layer (CoPW@NiO) with an electrochromic function.

Onto the transparent electrode of the transparent electrode/first electrochromic layer stack, BMIMTFSI (1-butyl-3-methylimidazolium bis(trifluoromethylsulfonyl)imide) ion gel including PVDF-HFP (M.W˜about 100,000) at 25 wt % is cut and attached without forming air bubbles to form an electrolyte layer and then, covered with the second electroactive layer with a CoPW@NiO electrochromic function in a sandwich form to manufacture an electrochromic device with an active area of about 1×1.5 cm².

### Comparative Example 1

An electrochromic device is manufactured in the same manner as in Example 1 except that the transparent electrode/electrochromic layer stack of Comparative Preparation Example 1 is used instead of the transparent electrode/electrochromic layer stack of Preparation Example 1.

### Evaluation I

The electrochromic devices according to Examples and Comparative Examples are evaluated with respect to electrochromic characteristics.

Herein, a voltage is applied to each of the electrochromic devices by using a Compactstat.h manufactured by Ivium Technologies, which is a potentiostat, and a Lambda 750 UV-Vis spectrophotometer manufactured by PerkinElmer is used to measure changes in light transmittance resulting from the electrochromic characteristics of the electrochromic devices.

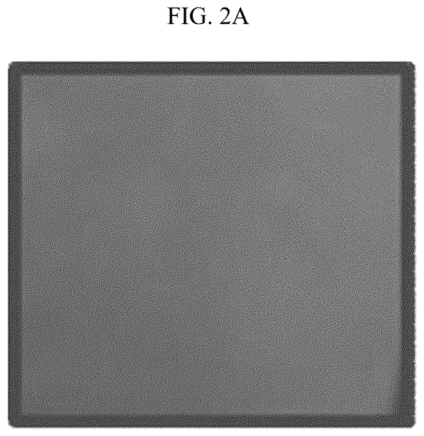

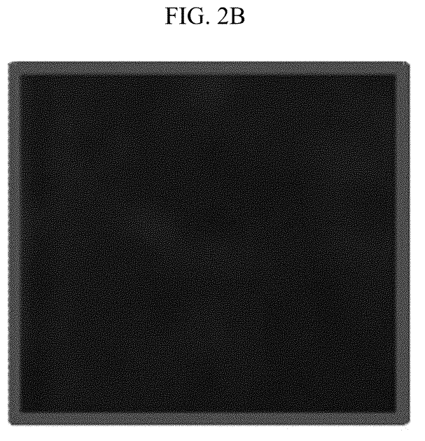

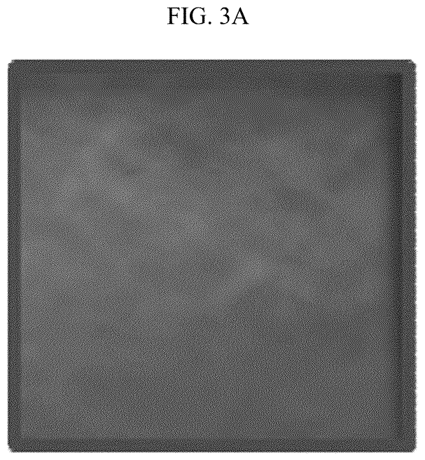

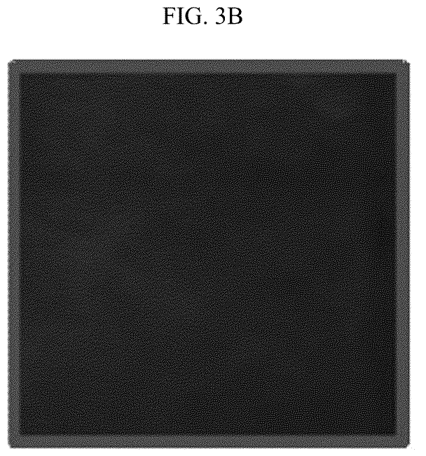

A constant current is input into the electrochromic devices of Examples and Comparative Examples in a chronoamperometry method, and simultaneously, transmittance output therefrom is monitored in real time by using UV-vis spectroscopy. The results are shown in FIGS. 2A, 2B, 3A and 3B.

FIGS. 2A and 2B are photographs showing the color change according to the voltage application of the electrochromic device according to Example 1, and FIGS. 3A and 3B are photographs showing the color change according to the voltage application of the electrochromic device according to Comparative Example 1.

Referring to FIGS. 2A and 2B, if-1 V is applied to the electrochromic device of Example 1, light transmittance at a wavelength of 500 nm is 5% (black, FIG. 2B), and if +2.5 V is applied thereto, the light transmittance at the wavelength of 500 nm is 71% (transparent, FIG. 2A), from which a color contrast is confirmed to be 66%. If-1 V and +2.5 V are sequentially repetitively applied to the electrochromic device of Example 1, black (FIG. 2B) and transparence (FIG. 2A) are repeatedly displayed, which may confirm electrochromic change repeatability.

In contrast, referring to FIGS. 3A and 3B, if-2 V is applied to the electrochromic device of Comparative Example 1, light transmittance at a wavelength of 500 nm is 20% (blue-violet, FIG. 3B), and if +2.5 V is applied thereto, the light transmittance at the wavelength of 500 nm is 78% (transparent, FIG. 3A), from which a color contrast is confirmed to be 58%.

Accordingly, the electrochromic device of Example 1, compared with the electrochromic device of Comparative Example 1, is confirmed to effectively realize black and transparence, and thus a higher color contrast.

### Evaluation II

The electrochromic devices of Examples are evaluated with respect to black characteristics during the coloring.

During the coloring, the color characteristics are compared by using a color coordinate based on CIE 1931.

In order to measure the color coordinate based on CIE 1931, after applying a voltage to the electrochromic devices, a color diagram of an electrochromic device maintaining the coloring is measured by using a chromaticity meter.

The results are shown in Table 1.

TABLE 1

| | Applied voltage (V) | R | G | B | X | Y |
|---|---|---|---|---|---|---|
| Example 1 | −3 | 17 | 18 | 33 | 0.25 | 0.23 |
| Example 3 | −3 | 38 | 28 | 32 | 0.34 | 0.31 |
| Example 5 | −1.5 | 26 | 23 | 29 | 0.30 | 0.28 |

Referring to Table 1, the electrochromic devices of Examples are confirmed to effectively realize black, and in particular, (0.34, 0.31) of Example 3 and (0.30, 0.28) of Example 5, which are a color coordinate closer to a black body, are confirmed to effectively realize black.

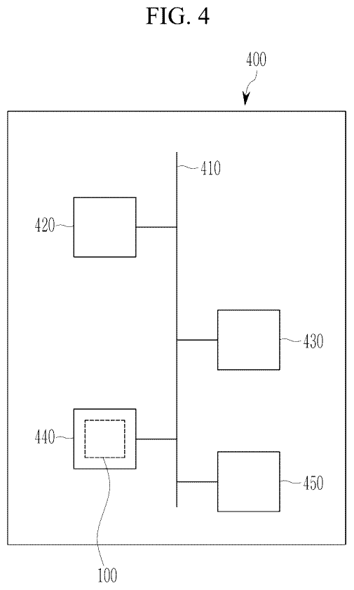

FIG. 4 is a schematic view of an electronic device according to some example embodiments.

Referring to FIG. 4, an electronic device 400 may include a processor 420 (e.g., a central processing unit or CPU), a memory 430 (e.g., a solid-state drive (SSD) storage device), an additional device 440, and a power supply 450 electrically connected to each other through a bus 410. The additional device 440 may include an electrochromic device 100 according to any of the example embodiments and examples described above (e.g., any one of the electrochromic devices in FIG. 1 and/or Examples 1 to 5). For example, the additional device 440 may include an optical filter, an electronic signboard, a smart window, a wearable imaging device and/or display device (such as an augmented reality (AR) or a virtual reality (VR) imaging device and/or display device), any combination thereof, or the like, any of which may include an electrochromic device 100 according to example embodiments. In some example embodiments, the additional device 440 may include a processor (e.g., a CPU) and/or a memory (e.g., an SSD). The memory 430 (and/or a memory of the additional device 440), which may be a non-transitory computer readable medium (e.g., an SSD storage device), may store a program of instructions. The processor 420 (and/or a processor of the additional device 440), for example a CPU, may execute the stored program of instructions to perform one or more functions. The power supply 450 may include a rechargeable battery, such as a lithium-ion battery. The processor 420 (and/or a processor of the additional device 440) may be configured to adjustably control a voltage of electrical power (e.g., electrical power supplied from the power supply 450) applied to one or more elements of an electrochromic device 100 of the additional device 440. For example, the processor 420 (and/or a processor of the additional device 440) may be configured to adjustably control a supply of electrical power from the power supply 450 to the additional device 440, for example to adjustably control a voltage applied to an electrochromic device 100 of the additional device 440, to control a color and/or opacity exhibited by at least an electrochromic layer 14 of the electrochromic device 100. The processor 420 (and/or a processor of the additional device 440) may be configured to generate an output (e.g., cause a certain voltage to be applied to the electrochromic layer 30 of the electrochromic device 100 of the additional device 440, to cause the electrochromic device 100 to exhibit a particular color and/or opacity) based on processing a program of instructions stored at the memory 430 (and/or a memory of the additional device 440). In some example embodiments, the additional device 440 may be electrically connected to the power supply 450 independently of a bus 410, and a processor included in the additional device 440 and/or the power supply 450 may be configured to adjustably control the application of voltage to the electrochromic device 100 based on electrical power supplied from the power supply 450. In some example embodiments, one or more of the bus 410, the processor 420, the memory 430, and/or the power supply 450 may be omitted.

The electronic device 400 and/or any portion thereof (e.g., processor 420, memory 430, additional device 440, power supply 450, etc.) may include processing circuitry such as a hardware including logic circuits; a hardware/software combination such as processor-implemented software; or any combination thereof. For example, the processing circuitry may be a central processing unit (CPU), an arithmetic logic unit (ALU), a digital signal processor, a microcomputer, a field programmable gate array (FPGA), System-on-Chip (SoC), a programmable logic unit, a microprocessor, an application-specific integrated circuit (ASIC), or the like. As an example, the processing circuitry may include a non-transitory computer readable storage device. The processor 420 (e.g., a central processing unit or CPU) may, for example, control an operation of the additional device 440 (e.g., an electrochromic device 100 thereof), for example based on executing an instruction program stored at the memory 430 (e.g., a solid-state drive (SSD) storage device).

While this disclosure has been described in connection with what is presently considered to be practical example embodiments, it is to be understood that inventive concepts are not limited to the disclosed embodiments. On the contrary, inventive concepts are intended to cover various modifications and equivalent arrangements included within the spirit and scope of the appended claims.

What is claimed is:

1. A compound represented by Chemical Formula 1: 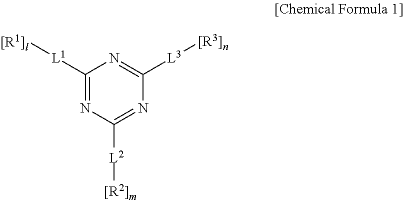
wherein, in Chemical Formula 1,
L¹ to L³ are each independently a single bond, O, S, NR^{a}, a substituted or unsubstituted C1 to C10 oxyalkylene group, a substituted or unsubstituted C1 to C10 thioalkylene group, a substituted or unsubstituted C6 to C30 arylene group, a substituted or unsubstituted divalent C3 to C30 heterocyclic group, or a combination thereof,
R^{a} is hydrogen, a substituted or unsubstituted C1 to C10 alkyl group, a substituted or unsubstituted C2 to C30 alkenyl group, a substituted or unsubstituted C2 to C30 alkynyl group, a substituted or unsubstituted C1 to C30 alkoxy group, a substituted or unsubstituted C6 to C30 aryl group, a substituted or unsubstituted C3 to C30 heteroaryl group, or a combination thereof,
I, m, and n are each independently an integer greater than or equal to 1,
R¹ to R³ are each independently a substituted or unsubstituted C6 to C30 aryl group, a substituted or unsubstituted C3 to C30 heterocyclic group, a substituted or unsubstituted amino group, or a combination thereof, provided that at least one of R¹ to R³ independently is a group represented by Chemical Formula A, 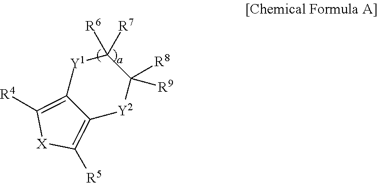
wherein, in Chemical Formula A,
X, Y¹, and Y² are each independently O, S, Se, or Te,
R⁴ to R⁹ are each independently hydrogen, a substituted or unsubstituted C1 to C30 alkyl group, a substituted or unsubstituted C2 to C30 alkenyl group, a substituted or unsubstituted C2 to C30 alkynyl group, a substituted or unsubstituted C1 to C30 oxyalkyl group, a substituted or unsubstituted C1 to C30 haloalkyl group, a substituted or unsubstituted C1 to C30 alkoxy group, a halogen, a cyano group, or a linking point with Chemical Formula 1, provided that at least one of R⁴ to R⁹ in Chemical Formula A are a linking point with Chemical Formula 1,
a is an integer from 1 to 3, and
if a is 2 or 3 in Chemical Formula A, R⁶ are the same as or different from each other and R⁷ are the same as or different from each other.

2. The compound of claim 1, wherein
one or two of L¹ to L³ is different from a remainder of L¹ to L³, or
one or two of R¹ to R³ is different from a remainder of R¹ to R³.

3. The compound of claim 2, wherein
the one or two of L¹ to L³ are each independently O, S, a substituted or unsubstituted C1 to C10 oxyalkylene group, or a substituted or unsubstituted C1 to C10 thioalkylene group, and
the remainder of L¹ to L³ are each independently NR^{a}, wherein R^{a} is hydrogen, a substituted or unsubstituted C1 to C10 alkyl group, a substituted or unsubstituted C2 to C30 alkenyl group, a substituted or unsubstituted C2 to C30 alkynyl group, a substituted or unsubstituted C1 to C30 alkoxy group, a substituted or unsubstituted C6 to C30 aryl group, a substituted or unsubstituted C3 to C30 heteroaryl group, or a combination thereof.

4. The compound of claim 2, wherein
the one or two of L¹ to L³ are each independently O, S, or NR^{a}, wherein
R^{a} is hydrogen, a substituted or unsubstituted C1 to C10 alkyl group, a substituted or unsubstituted C2 to C30 alkenyl group, a substituted or unsubstituted C2 to C30 alkynyl group, a substituted or unsubstituted C1 to C30 alkoxy group, a substituted or unsubstituted C6 to C30 aryl group, a substituted or unsubstituted C3 to C30 heteroaryl group, or a combination thereof, and
the remainder of L¹ to L³ are each independently a substituted or unsubstituted C1 to C10 oxyalkylene group or a substituted or unsubstituted C1 to C10 thioalkylene group.

5. The compound of claim 2, wherein R¹ to R³ are each different from each other.

6. The compound of claim 1, wherein the group represented by Chemical Formula A is represented by Chemical Formula A-1 or Chemical Formula A-2: 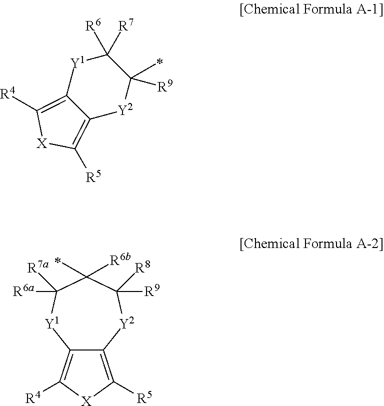
wherein, in Chemical Formula A-1 or A-2,
X, Y¹, and Y² are each independently O, S, Se, or Te,
R⁴, R⁵, R⁶, R^{6a}, R^{6b}, R⁷, R^{7a}, R⁸, and R⁹ are each independently hydrogen, a substituted or unsubstituted C1 to C30 alkyl group, a substituted or unsubstituted C2 to C30 alkenyl group, a substituted or unsubstituted C2 to C30 alkynyl group, a substituted or unsubstituted C1 to C30 oxyalkyl group, a substituted or unsubstituted C1 to C30 haloalkyl group, a substituted or unsubstituted C1 to C30 alkoxy group, a halogen, a cyano group, or a combination thereof, and
* is a linking point with Chemical Formula 1.

7. The compound of claim 6, wherein, in Chemical Formula 1,
some of R¹ to R³ is a group represented by Chemical Formula A-1, and
a remainder of R¹ to R³ is a group represented by Chemical Formula A-2.

8. The compound of claim 6, wherein, in Chemical Formula 1,
R¹ is a group represented by Chemical Formula A-1,
R² is a group represented by Chemical Formula A-2, and
R³ is a substituted or unsubstituted phenyl group, a substituted or unsubstituted biphenyl group, a substituted or unsubstituted naphthyl group, a substituted or unsubstituted anthracenyl group, a substituted or unsubstituted fluorenyl group, a substituted or unsubstituted triphenylene group, a substituted or unsubstituted carbazolyl group, a substituted or unsubstituted aniline group, a substituted or unsubstituted pyrrole group, a substituted or unsubstituted indole group, a substituted or unsubstituted thiazole group, a substituted or unsubstituted benzothiazole group, a substituted or unsubstituted thiadiazole group, a substituted or unsubstituted benzothiadiazole group, a substituted or unsubstituted imidazole group, a substituted or unsubstituted benzoimidazole group, a substituted or unsubstituted pyrazolyl group, a substituted or unsubstituted phenylenevinylene group, a substituted or unsubstituted triphenylamine group, or a combination thereof.

9. The compound of claim 1, wherein, in Chemical Formula 1,
one or two of R¹ to R³ is a substituted or unsubstituted phenyl group, a substituted or unsubstituted biphenyl group, a substituted or unsubstituted naphthyl group, a substituted or unsubstituted anthracenyl group, a substituted or unsubstituted fluorenyl group, a substituted or unsubstituted triphenylene group, a substituted or unsubstituted carbazolyl group, a substituted or unsubstituted aniline group, a substituted or unsubstituted pyrrole group, a substituted or unsubstituted indole group, a substituted or unsubstituted thiazole group, a substituted or unsubstituted benzothiazole group, a substituted or unsubstituted thiadiazole group, a substituted or unsubstituted benzothiadiazole group, a substituted or unsubstituted imidazole group, a substituted or unsubstituted benzoimidazole group, a substituted or unsubstituted pyrazolyl group, a substituted or unsubstituted phenylenevinylene group, a substituted or unsubstituted triphenylamine group, or a combination thereof.

10. A polymer, the polymer comprising:
a structural unit derived from the compound of claim 1.

11. The polymer of claim 10, further comprising:
a moiety derived from polyethylene glycol, polypropylene glycol, or a combination thereof.

12. The polymer of claim 10, wherein the polymer includes at least one of R¹, R², or R³ of Chemical Formula 1 as a repeating unit.

13. A polymer film comprising:
the polymer of claim 10.

14. The polymer film of claim 13, wherein
the polymer film is configured to reversibly display black or transparency depending on an applied voltage, and
a difference in light transmittance depending on the applied voltage is greater than or equal to about 60% for light of a wavelength of about 500 nm.

15. An electronic device comprising:
the polymer film of claim 13.

16. An electrochromic device, the electrochromic device comprising:
a first electrode;
a second electrode;
an electrochromic layer between the first electrode and the second electrode;
the electrochromic layer comprising the polymer of claim 10; and
an electrolyte in contact with the electrochromic layer.

17. The electrochromic device of claim 16, wherein
the polymer comprises a first polymer and a second polymer, and
the first polymer and the second polymer include different repeating units from each other.

18. The electrochromic device of claim 17, wherein the electrochromic layer comprises
a first electrochromic layer including the first polymer, and
a second electrochromic layer including the second polymer.

19. The electrochromic device of claim 16, wherein
the polymer is configured to reversibly display black or transparency depending on an applied voltage, and
a difference in light transmittance depending on the applied voltage is greater than or equal to about 60% for light of a wavelength of about 500 nm.

20. An electronic device comprising:
the electrochromic device of claim 16.
